(19) United States
(12) Patent Application Publication
(10) Pub. No.: US 2004/0111034 A1
(43) Pub. Date: Jun. 10, 2004

(54) System for measuring at least one body parameter, a blood pressure monitor and a medical thermometer

(76) Inventors: Kin Yuan Lin, Taipei (TW); Ruey-Ting Chang, Taipei (TW)

Correspondence Address: SHOEMAKER AND MATTARE, LTD, P.O. BOX 2286, 2001 JEFFERSON DAVIS HIGHWAY, ARLINGTON, VA, 22202-0286, US

(21) Appl. No.: 10/433,475

(22) PCT Filed: Dec. 28, 2001
(86) PCT No.: PCT/EP01/15359

(30) Foreign Application Priority Data
Jan. 3, 2001 (US) 60259179
Jan. 16, 2001 (EP) 01100892.7
Sep. 10, 2001 (EP) 01121541.5

(51) Int. Cl. A61B 5/02

(52) U.S. Cl. 600/485; 600/500; 600/300; 128/903; 128/920; 600/549

(57) ABSTRACT

A system for measuring the value of at least one parameter and for processing or storing these values is provided with a body parameter measuring device and a computer program product. The body parameter measuring device has a sensor for measuring the body parameter and a processing unit for treating an electrical signal corresponding to this parameter. The body parameter measuring device is provided with a communication interface for communication with an external calculating device. The computer program product is used to cause the external calculating device to receive data from the body parameter measuring device and to store or treat this data.

The invention relates to a system for measuring the value of at least one body parameter, to a blood pressure monitor and to a medical thermometer according to the preamble of the independent patent claims. Devices for automatically measuring body parameters such as the blood pressure, the body temperature or the like are known in a wide variety. Such devices generally are provided with a micro processor which allows to treat and/or display the values of the body parameter on a display. It is e.g. known to calculate and display average values of body parameters such as a blood pressure in an automatic blood pressure monitor. Due to the limited calculation capacity of microprocessors used in such systems, the possibility of treatment of data is rather limited. In addition, such conventional body parameter measuring devices such as blood pressure monitors or medical thermometers should have as little operation buttons as possible in order to avoid confusing the user of such devices. Furthermore, the storage capacity of such devices is often limited. The possibility of data storage and data treatment in such devices is therefore, although desirable in many cases, rather limited.

It is therefore an object of the present invention, to provide a system for measuring the value of at least one body parameter which allows flexible treatment and/or storage of the values of these body parameters.

The object of the present invention is solved with a system according to the characterising part of the independent patent claims. The system according to the invention comprises at least one body parameter measuring device and a computer program product associated therewith. The body parameter measuring device has at least one sensor for providing an electrical signal corresponding to the at least one value of the body parameter. The measuring device further has a processing unit for treating the electrical signal. The system also includes a communication interface for communication of the body parameter measuring device with an external calculating device separate from the measuring device.

The computer program product has a computer usable medium having computer readable code means embodied therein. The code means have a series of computer readable program code means for causing the external calculating device to receive data from the body parameter measuring device and to store and/or treat said data in the calculating device.

By providing a body parameter measuring device with a communication interface and a computer program product which can be run on an external calculating device able to communicate with the body parameter measuring device, the invention allows to store or treat data relating to body parameters in a wide variety of applications. The possibility of data storage or data treatment is not limited by the body parameter measuring device. All data treatment or data storage can be made in the external calculating device. Typical body parameters are the body temperature, the blood pressure, the weight, glucose level or the peaks flow.

The use of an external calculating device and of a separate computer program product allow the implementation of a plurality of different data processing modes. According to a first embodiment of the invention, the program code means can cause the external calculating device to remind the patient to follow a predetermined measurement schedule. This can be done e.g. by creating an audible or visible signal. The signal can be created according to a measurement schedule stored in the calculating device. The measurement schedule can be checked when the external calculating device is started and thereafter on a regular basis, e.g. every 15 minutes.

According to another embodiment of the present invention, the program code means may cause the external calculating device to communicate with a central storage means or with a central server. Such communication may be made with the purpose of sending data to the central storage means or to the central server. It is desirable to store the values of body parameters over a long period of time. Especially doctors need to have access to the values of body parameters of their patients in the patient history. It is, however, difficult to store data in the measuring device over a long period of time and to allow access to such data from an external site. The external calculating device which may also have a limited storage capacity may therefore be used as a transmitter for forwarding data from the body parameter measuring device to a central storage unit. In this case, it is not necessary to provide the body parameter measuring-device with a separate communication interface for communication with the central storage means.

In a preferred embodiment of the present invention, the system comprises computer readable code means which are written such as to run on a personal digital assistant. Personal digital assistants are widely used today. A personal digital assistant is a very convenient external calculating device for the present invention. Conventional personal digital assistants are provided with a communication interface which allows easy communication with the body parameter measuring device. Such personal digital assistants have sufficient calculating capacity for data treatment. On the one hand, personal digital assistants allow for a very easy operation due to their graphical user interface. On the other hand, personal digital assistants also allow an easy communication with a central server or with the internet, so that data received from the body parameter measuring device can be easily transmitted to a central storage means.

According to a further preferred embodiment of the invention, the body parameter measuring device is a measuring device selected from the group consisting of blood pressure monitors, body weight meters, body fat meters, cholesterol meters, glucose meters, peak flow meters or clinical thermometers. Of course, more than one measuring device may be used in a system according to the present invention. The external calculating device allows to receive, treat and store data from any type of these measuring devices. The external calculating device therefore may become a kind of health centre for the user, in which a plurality of different body parameters may be stored. The use of a personal digital assistant as a personal health centre is particularly preferred.

According to a further embodiment of the invention, the program code means cause the external calculating device to display a graphic representation of the values of the body parameter. The graphic representation is advantageous because the user can more easily follow the historical behaviour of the health parameter than only by referring to numerical values. The possibilities of a numerical LCD display used in a conventional body parameter measuring devices is rather limited. The use of an external calculating device allows to produce a variety of different graphical representations, even in colour if the external calculating device allows so. It will be understood that the use of a personal digital assistant is advantageous in this context, as personal digital assistants may have a graphical colour display.

According to a further preferred embodiment of the invention, the program code means may cause the external calculating device to ask for an input of patient data. It is conceivable to ask e.g. for the name of the user, but also for other patient data such as age, weight, sex or the like. By providing such an input, a plurality of further data treatment is possible. As the external calculating device, e.g. a personal digital assistant may be provided with a graphical user interface for data input, input of data can be easily made. No additional data input means on the body parameter measuring device are necessary.

According to a first preferred embodiment of the invention, the body parameter measuring device is a blood pressure monitor. The program code means cause the external calculating device to store a plurality of subsequent pulse rate values or blood pressure values. Such a system can be e.g. used for stratification of risk factors. In a further preferred embodiment of the present invention, the program code means therefore can cause the external calculating device to provide a user input interface for entering relevant data in context with the risk to develop a disease such as a cardiovascular disease. Such data are representative for specific risk factors. The program code means thereafter cause the external calculating device to store these data in a memory and to calculate a risk rate on the basis of these risk factors and of the measured blood pressure values or the pulse rate. The risk rate as calculated is thereafter displayed. A personal digital assistant as a external calculating device has all components needed for such a system: The PDA is especially provided with an input interface and with a display. The PDA is further powerful enough to make such calculations and has sufficient storage capacity to store such risk factors. The risk factors are selected from the group consisting of sex and age, smoker, cholesterol level, family history and diabetes according to a preferred embodiment of the invention.

According to a further preferred embodiment of the invention, the program code means cause the external calculating device to calculate and display a limited number of different risk rates such as NO RISK, LOW RISK, MEDIUM RISK, HIGH RISK and VERY HIGH RISK. The system according to the present invention usually includes one or more body parameter measuring devices and a computer program product associated therewith. It is also possible to include the external calculating device in the system. Complete systems consisting of e.g. a blood pressure monitor, a medical thermometer, a computer program product and a personal digital assistant on which the computer program product can be run are e.g. conceivable.

According to a further preferred embodiment, the program code means allows the external device to permanently store data relating to plurality of users. The external calculating device, e.g. a personal digital assistant, may therefore be used as a health centre for a plurality of persons, e.g. for a family. In this context, data relating to separate users may be password protected.

Conventional personal digital assistants are usually provided with an infrared (IrDA) interface. According to a further preferred embodiment, the system according to the present invention includes a body parameter measuring device which is also provided with an IrDA interface. This allows very easy communication with standard calculating devices. Instead of communications with PDA's, communication with portable computers such as conventional Laptops (which are also provided with IrDA interfaces) is possible with an appropriate computer program product.

According to another aspect of the invention, there is provided a blood pressure monitor and a computer program product associated therewith. The computer program product can be run on the blood pressure monitor or on a separate calculating device. Especially, the blood pressure monitor can be used in a system as described above. The computer program product has a computer usable medium having computer readable code means embodied therein. The program causes the blood pressure monitor or the external calculating device to store the measurement results after each measurement. After each measurement, the program code means cause the blood pressure monitor or an external calculating device to form an average or averages of pulse rate values or blood pressure values stored in the blood pressure monitor or in the external calculating device. These values are thereafter compared with the predetermined first threshold value or with predetermined first threshold values. Thereafter, the program code means cause the blood pressure monitor or the external calculating device to compare all of the actual measurement-result with a second threshold value or with second threshold values. An indication that the patient should take medicine is displayed when either the average is above the first threshold values or when at least one of the values is above the second threshold values. Such comparison may be made e.g. for the values of the systolic, the diastolic blood pressure and fore the pulse rate and for the respective averages thereof. Obviously, separate threshold values are applicable for each of the values of the diastolic, the systolic blood pressure and the pulse rate.

According to an alternative embodiment of the invention, the body parameter measuring device is a medical thermometer. The program code means cause the external calculating device to display e.g. a series of temperature values. Such a system may be used e.g. for natural family planning by way of the symptothermal method. Temperature values measured during the e.g. last thirty days are displayed on the external calculating device. A graphic display of the temperature allows easier observation of temperature changes, especially increases.

The medical thermometer used in a system according to the present invention can be specifically designed. It may comprise a housing and a stem projecting from the housing. A tip with a temperature sensor is arranged on the stem. The stem is made of a flexible material and is adapted to be wound around the housing. Such a thermometer can be easily stored, e.g. in a bag or in a pocket. The housing is provided with a communication interface with the external calculating unit. It is therefore not necessary to provide a display on the medical thermometer. A very compact thermometer design therefore is possible. Such design is, however not limited to a system described before.

The invention will be understood in more detail with reference to the following embodiments and drawings which show:

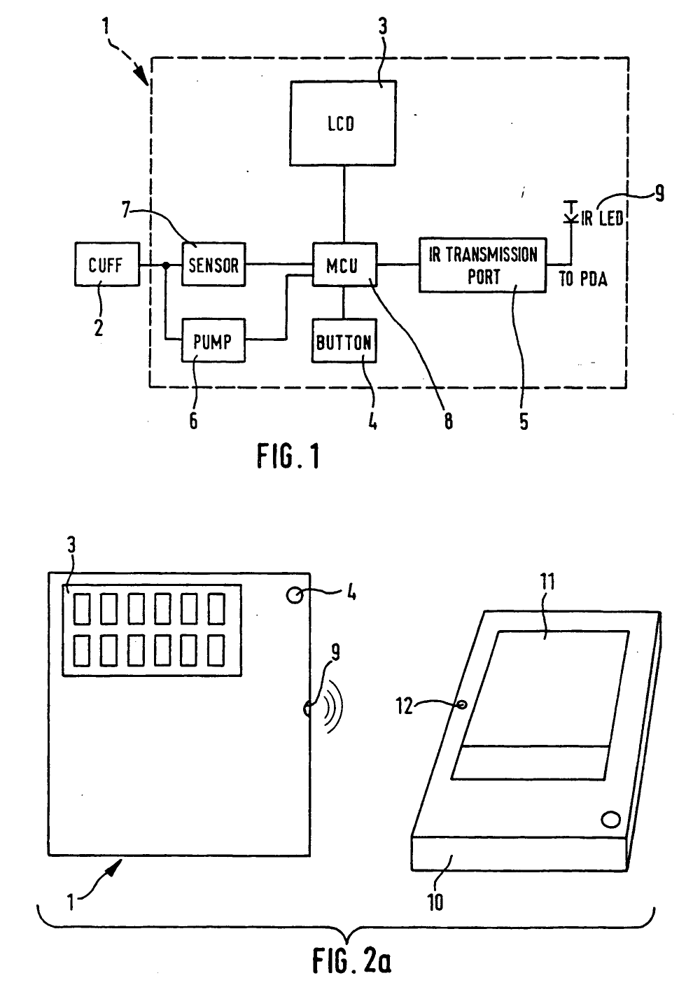

FIG. 1: A schematic representation of a blood pressure monitor in a system according to the invention,

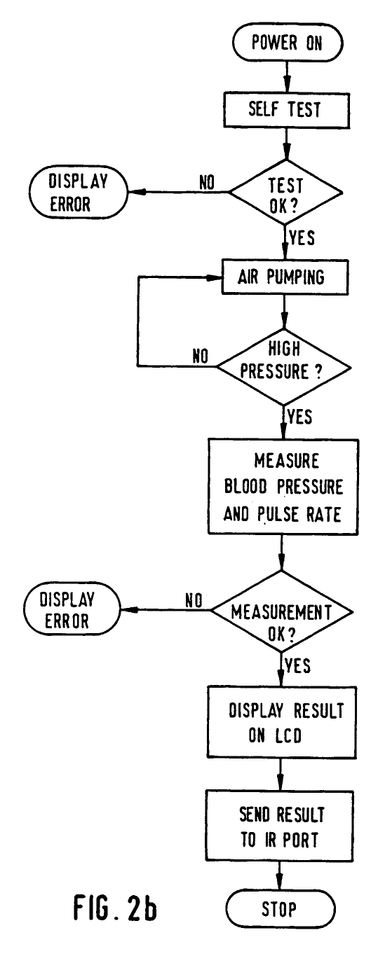

FIG. 2a: a schematic representation of a blood pressure monitor and of a personal digital assistant according to the invention,

FIG. 2b: a flow chart showing a measurement cycle in a blood pressure monitor,

FIG. 2c: a flow chart showing the reading of data into an external calculating device,

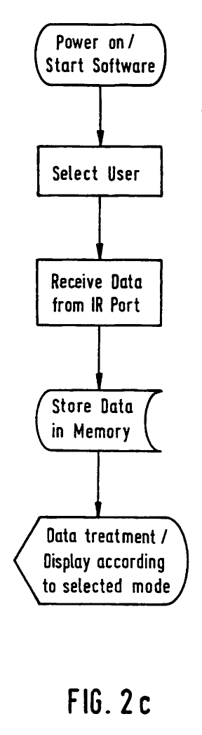

FIG. 3: a flow chart showing a first specific form of data treatment,

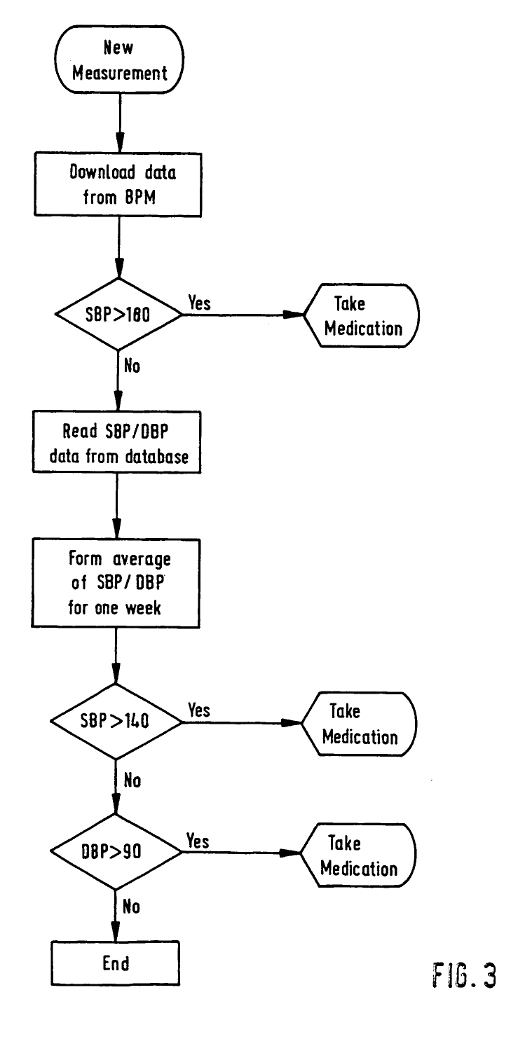

FIG. 4a: a schematic representation of a graphical display of blood pressure values,

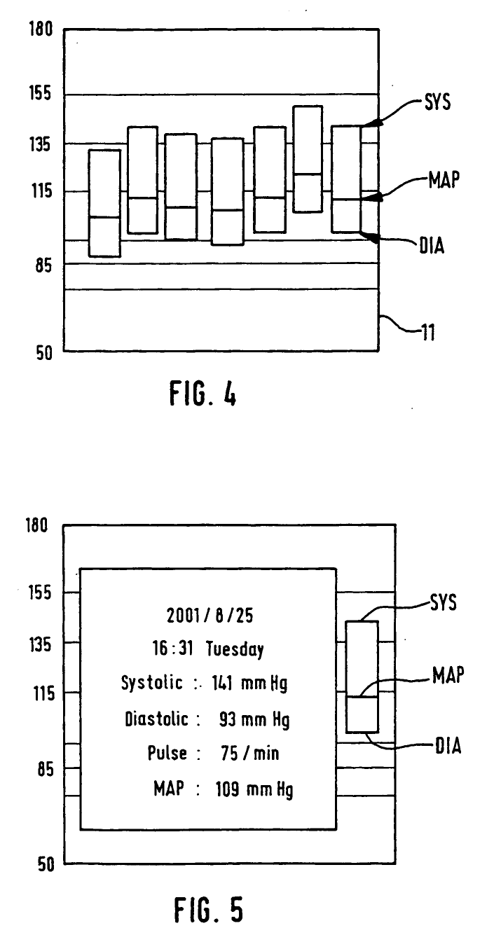

FIG. 5: a schematic representation of the display of the result of one measurement,

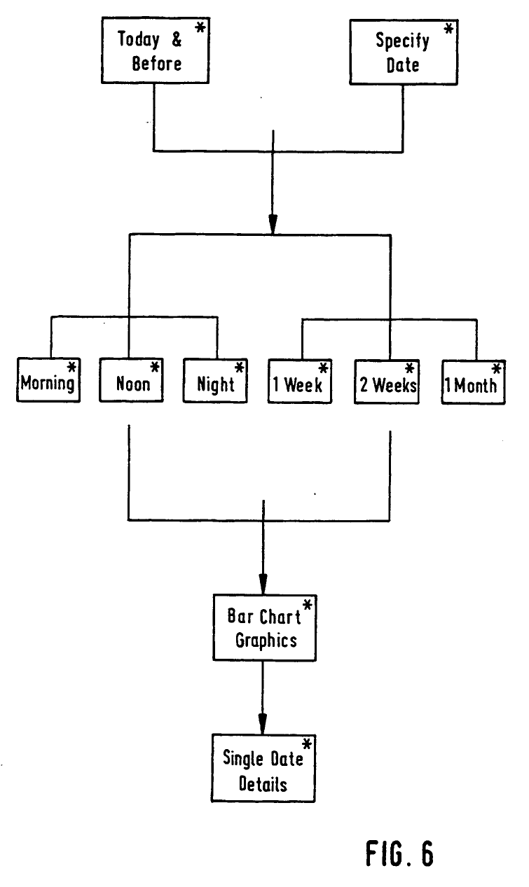

FIG. 6: a flow chart showing method steps for data representation,

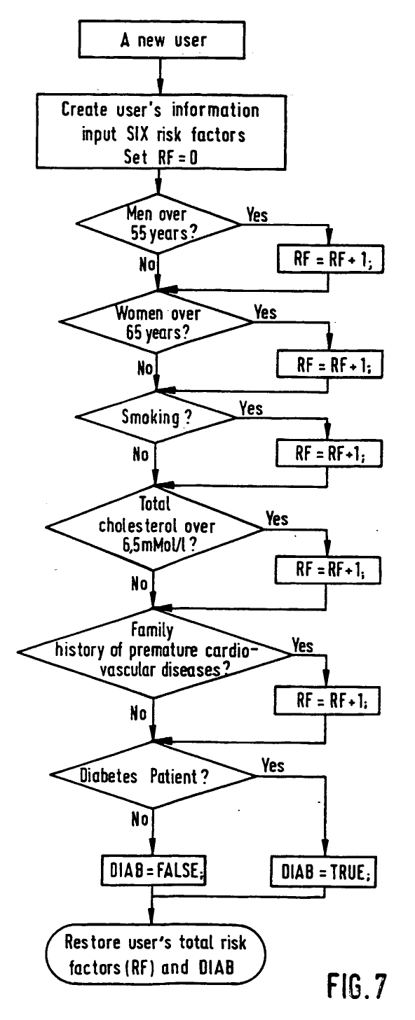

FIG. 7: a flow chart showing the steps for creating a risk profile,

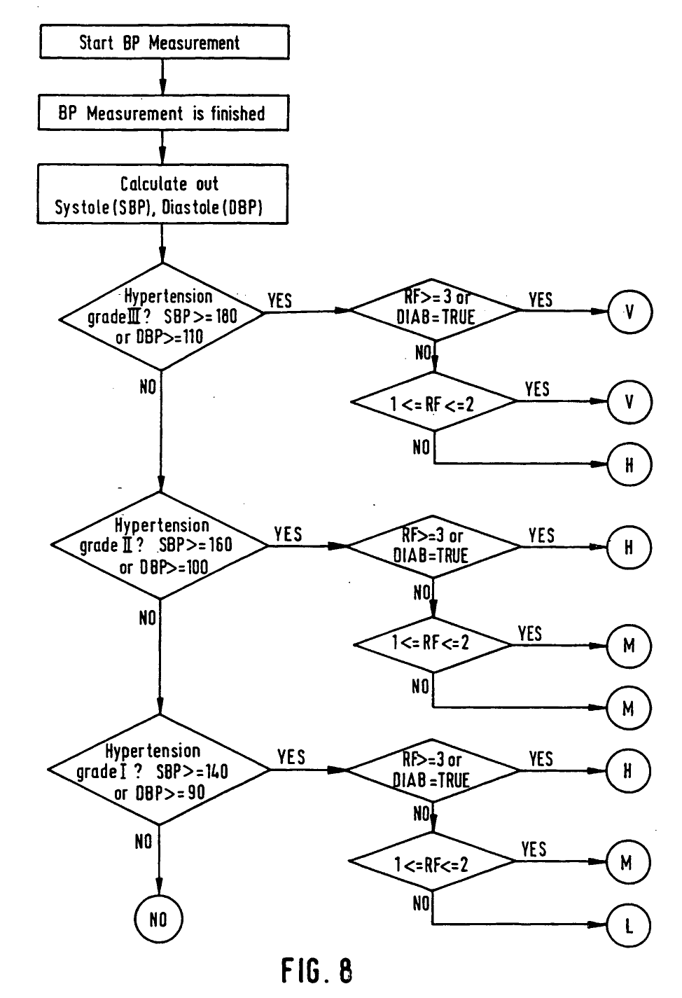

FIG. 8: a flow chart showing the calculating operation for determining a risk rate,

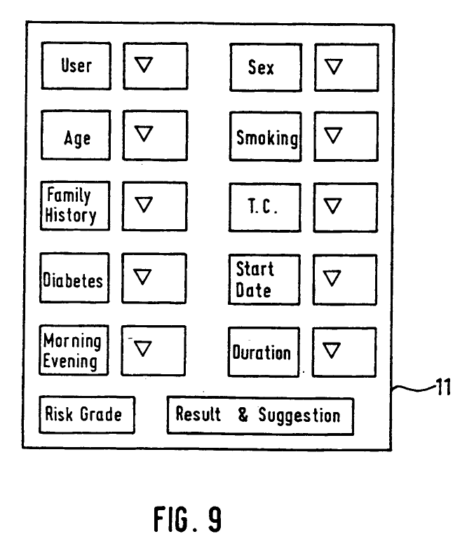

FIG. 9: a schematic representation of a graphical user input interface and

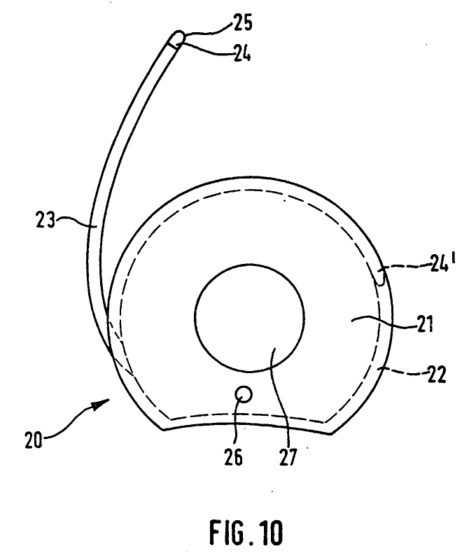

FIG. 10: a schematic representation of a thermometer according to the invention.

FIG. 1 shows a blood pressure monitor 1 for use in a system according to the invention. The blood pressure monitor 1 comprises a cuff 2 which can be wound around a patient's upper arm or a patient's wrist. The blood pressure monitor 1 is provided with a display 3 on which measurement results can be displayed. The blood pressure monitor 1 is further provided with one single button 4 allowing to turn the blood pressure monitor on/off. When the start button 4 is pressed, a pump 6 starts to inflate the cuff 2 to a desired pressure level in an known manner. The pressure in the cuff 2 is measured with a sensor 7. A measurement of the blood pressure is made by acquisition of a pulse wave signal corresponding to the pressure in the cuff 2 during deflation of the cuff 2. The operation of the blood pressure monitor is controlled by a main calculating unit 8. The main calculating unit 8 especially starts the pumping and measuring process when the button 4 is pressed. The main calculating unit 8 also is used to calculate blood pressure values, especially the diastolic, the systolic blood pressure and the main arterial blood pressure and the pulse rate on the basis of the pulse ware signal. The values are then displayed on a display 3 and at the same time transferred to a infrared transmission port 5. The blood pressure monitor is provided with an infrared LED 9 allowing data exchange with other devices such as a personal digital assistant (PDA) 10.

FIG. 2a schematically shows a system-according to the invention. The system consists of a blood pressure monitor 1 and a personal digital assistant 10. Measurement results are displayed on display 3 of the blood pressure monitor. The results are also transmitted via a infrared communication link by means of an infrared LED 9. Data transmitted from the blood pressure monitor 1 are received in the personal digital assistant by an infrared receiver 12. The display 11 of the personal digital assistant can be used for any type of display of data or input of patient specific data. Instead of IR data transmission, other transmission modes such as wire transmission are possible.

The steps carried out during measurement are shown in the flow chart according to FIG. 2b. As soon as the power is switched on by pressing the button 4, a self test is carried out. If the test fails, an error message is displayed. If the test is passed, the pump 6 starts to pump air into the cuff 2 until a sufficiently high pressure is achieved: When this pressure is achieved, the measurement of the blood pressure and of the pulse rate is started. The measurement is made in the osillometric method which is well known to those skilled in the art. At the end of the measuring step, the correctness of the measurement may be verified. If this test is failed, an error message is displayed. If the test is passed, the measurement result is displayed on the display 3 and the results are sent to the infrared transmission port 5. After displaying and transmitting the measurement results, the blood pressure monitor 1 is either automatically stopped after a certain period of time or turned off by pressing the on/off button 4. On/off button 4 is the only operating key of the blood pressure monitor 1. User errors therefore can be greatly reduced.

FIG. 2c shows the steps which are carried out by the personal digital assistant 10. After start of the personal digital assistant, a specific computer program is loaded in the personal digital assistant 10. The computer program is specifically designed for communication with a body parameter measuring device such as the blood pressure monitor 1 or a medical thermometer 20 (see FIG. 10). After start of the software program, a user selection is possible. The software can be designed either for single user or for multi user operation. In the case of a single user operation, no user selection is necessary. In the case of a multi user operation, the user may be selected among a plurality of users profiles stored in the personal digital assistant. Storage of user profiles may be made in a separate method step in a manner known to those skilled in the art. In this context, it is conceivable to store the name of the user together with health data such as sex, weight, age or other specific parameters. It is also conceivable to provide the user selection with a password in order to ensure privacy of the stored data.

After user selection, data sent from the blood pressure monitor 1 or from other body parameter measuring devices are received by means of an infrared port of the personal digital assistant 10. The received data are subsequently stored in a database in a memory of the personal digital assistant. On the basis of the data stored in the memory, a data treatment or a data display according to one or more selected operating modes is carried out.

FIG. 3 shows schematically the steps of a first operating mode. This operating mode is used to determine whether a user should take medication. In a first step, it is verified whether the systolic blood pressure received from the blood pressure monitor is above 180 mm/hg. If yes, a message informing the user, that he should take his medication is displayed on display 11. If no, the values of systolic and diastolic blood pressures of previous measurements stored in the data base are read. An average of the systolic blood pressure and of the diastolic blood pressure of a plurality of subsequent measurements, e.g. all measurements made within one week, is formed thereafter. If either the average of the systolic blood pressure is above 140 mm/hg or average of the diastolic blood pressure is above 90 mm/hg, a message informing the user that he should take his medication is displayed. If neither the systolic blood pressure is above 140 mm/hg in average nor the diastolic blood pressure is above 90 mm/hg in average, this procedure is stopped and no separate display is made. Of course, it is conceivable to display the current values and/or the average values on the display of the personal digital assistant.

The personal digital assistant also allows to graphically display the measurement results. FIG. 4 shows a possible representation mode. The measurement results of seven subsequent measurements, e.g. one measurement per day during one week, are displayed on display 11 of the personal digital assistant 10. The values of the diastolic blood pressure, the systolic blood pressure and the mean arterial pressure are displayed as bar charts. This graphic representation allows the user to easily follow the behaviour of his blood pressure and to observe any tendencies or changes.

In FIG. 5 there is shown a combination of a numeric and graphic representation of the measurement results. Together with the day and date of the measurement, the values of the systolic and the diastolic blood pressure, the mean arterial blood pressure and the pulse rate are indicated. The blood pressure values are also displayed as a bar chart. The way of displaying graphics is known to those skilled in the art and does not need to be explained in detail. A plurality of different representation modes, e.g. display of daily, weekly, monthly measurements is conceivable.

FIG. 6 shows a flow chart for selecting a specific representation mode. In a first step, the date of the measurement is chosen. The user can either specify a specific date or select the present day. In a next step, the measurement time (morning/noon night) may be selected. Additionally, the period of display (one week/two weeks/one month) may be selected. Depending on the selection previously made, a bar chart graphics is calculated on the basis of the measurement values stored in the memory of the personal digital assistant. After the display of the bar chart graphics, single details for each measurement may be displayed, e.g. when the user clicks on a specific bar in the representation as shown in FIG. 4.

FIGS. 7 and 8 show a further possible operation mode in which a risk for suffering from a specific disease is calculated.

In a first step, risk factors for a user are defined. The procedure for entering the risk factors is shown in the flow chart of FIG. 7. At the beginning of the set up procedure for a new user, the total risk factor RF is set to 0. Thereafter the user is asked, whether he is a man over 55 years. If the answer is “Yes” i.e. if a OK button is clicked on on the display, the counter for the total risk factor RF is increased by 1. If the answer is “No”, the user is asked, whether he or she is a woman above 65 years. If the answer is “Yes”, the counter for the total risk factor is increased by 1.

The user is then asked if he or she is a smoker. If “Yes”, the counter for the total risk factor RF is increased by 1.

The user is then asked, whether his or her total cholesterol level is above 6.5 mMol per litre (250 mg per dl). If the answer is “Yes”, the counter for the total risk factor RF is again increased by 1.

The user is then asked, whether there is a family history of premature cardiovascular diseases. Again, if the answer is “Yes”, the counter for the total risk factor RF is increased by 1.

The user is then asked, whether he is suffering from diabetes. If the answer is “Yes”, a variable DIAB is set as “true”. If the answer is “No”, the variable DIAB is set to “false”.

At the end of the procedure as shown in FIG. 2, the total risk factor RF and the variable DIAB are stored in the memory of the PDA 10.

The risk factors may be stored over a longer period of time. A different risk factor profiles may be stored for a number of users. In this case, before starting the set up procedure as shown in FIG. 7, a new user must be created.

After the set up procedure for setting up risk factors, the total risk factor has a value between 0 and 4 and the variable DIAB has either a value of “false” or “true”.

FIG. 8 shows a flow chart showing the method of calculating a risk rate indicating to the patient his risk of suffering from a cardiovascular disease.

At the beginning, there may be a possibility for the user to select stored risk factor profiles or to define a new risk factor profile in accordance with FIG. 7. The values of the systolic and the diastolic blood pressure are read from the database or the PDA.

After the end of reading step, a classification of the blood pressure values into three categories, i.e. hypertension grade I, grade II and grade III is made. If the systolic blood pressure is above a first limit of 180 or the diastolic blood pressure is above a first limit of 110, the patient is classified as a hypertension grade III patient.

If the systolic blood pressure is above a second limit of 160 or the diastolic blood pressure is above a second limit of 100, the patient is classified as hypertension grade II.

If the systolic blood pressure is above a third limit of 140 or the diastolic blood pressure is above a third limit of 90, the patient is classified as hypertension grade I.

Calculation of a risk rate is made as follows with reference to FIG. 8:

If the patient is classified as grade III, it is checked, whether the total risk factor RF is equal or greater than 3 or if the variable DIAB was previously set to “true”. If “Yes”, a risk rate V indicating a very high risk is displayed in the section RR of the display 11 of the PDA. If the check is “No”, it is verified, whether the total risk factor RF is equal to 1 or 2. If the answer is “Yes”, a risk rate V indicative of a very high risk is displayed. If the answer is “No”, the risk rate symbol H is displayed, which indicates high risk.

If the patient is not classified as grade III and is classified as grade II, the same tests as above is made. If the total risk factor RF is greater than or equal to 3 or if the variable DIAB is set to “true”, the result of the calculation is a risk factor H indicating a high risk. If the total risk factor RF is 1 or 2, a risk rate M indicating a medium risk is displayed on the display 11. If the total risk factor is 0, the risk rate M is displayed.

If the patient is not classified as grade II, it is tested, whether he or she is classified as grade I. If the answer is “Yes”, the same tests as described above are carried out. If the total risk factor RF is above or equal to 3, or if the variable DIAB is “true”, a risk rate H is displayed. If the risk factor is 1 or 2, a risk rate M indicating a medium risk is displayed. If the risk factor is 0, a risk rate L indicating a low risk is displayed.

If the patient is not classified as grade I, i.e. if both, the systolic and the diastolic values are below the third limit, i.e. below 140 and 90 respectively, the system assumes that the risk of the patient of suffering from a cardiovascular disease is very low, so that no indication of a risk rate will be made. In this event, no symbol is displayed on the display 11.

The blood pressure monitor according to the invention allows a stratification of risk for a specific patient suffering from a cardiovascular disease. The stratification is made according to the guidelines of 1999 of the World Health Organisation International Society of Hypertension for the management of hypertension. The system allows an easy stratification of risk rates.

FIG. 9 shows a possible graphic user interface on the display 11 allowing the user to enter risk factors. The risk grade determined as described above can also be displayed on the display 11. It is further conceivable to display specific results or suggestions in view of a healthier life style of the patient.

FIG. 10 schematically shows an alternative embodiment of a body parameter measuring device in a system according to the invention. According to FIG. 10, a medical thermometer 20 with a specific design is provided. The medical thermometer 20 comprises a body 21 which is formed as a substantially round part. The body 21 is provided with a groove 22 running along its periphery. A stem 23 made of a flexible material is connected to the body 21. The stem is provided with a metal tip 24 at one end located opposite the connection of the stem 23 to the body 21. A temperature sensor 25 is provided in the metal tip 24. The flexible stem can be wound around the body 21 and can be placed in the groove 22. Reference numeral 24′ shows in dashed line the tip of the medical thermometer, when the stem 23 is wound around the body.

The medical thermometer 20 is provided with an infrared LED for transmission of data to an external calculating device, such as a personal digital assistant 10. The medical thermometer is further provided with a start or on/off button 27. The use of a thermometer in context with an external calculating device has several advantages. Especially, such a design allows an easy use of the thermometer for family planning. If the temperature is measured each day at the same time, the measurement result is transmitted to the personal digital assistant, where it is stored. The temperature values of each day in a certain period of time, e.g. within the last 30 days, can be displayed on the external calculating device. Especially, the use of such an external calculating device allows a graphic display of the values. It is not necessary to compare numerical values for detecting an increase in temperature. By simply observing the temperature curve, temperature increases may be easily detected. The thermometer shown in FIG. 10 is very convenient to carry e.g. in a bag or in a pocket due to its small size and due to its flexible stem. The flexible stem can be made e.g. of silicone. The thermometer according to FIG. 10 can be provided with a separate display. This is, however, not necessary as temperature values are directly transmitted to and stored in the personal digital assistant. Such a thermometer is also helpful in other context than in a system according to the present invention, e.g. as a stand alone temperature measurement unit.

It is known that personal digital assistants can be easily connected to a remote server, e.g. to the internet by means of a mobile phone. Personal digital assistants are usually provided with such communication possibilities. The personal digital assistant according to the invention can also be used for data transmission to a central server, e.g. a central health management system. For this purpose, the software which is run on the personal digital assistant further allows data transmission via a mobile phone to a central server, e.g. by using data transfer on the basis of TCP/IP.

1. A system for measuring the value of at least one body parameter and for processing and/or storing this at least one value,
the system comprising at least one body parameter measuring device and a computer program product,
wherein said body parameter measuring device has
at least one sensor for providing an electrical signal corresponding to said at least one value of the body parameter,
a processing unit for treating said electrical signal and
a communication interface for communication of said body parameter measuring device with an external calculating device separate from the body parameter measuring device
and wherein the computer program product has a computer usable medium having computer readable code means embodied therein, said code means having a series of computer readable program code means for causing said external calculating device to receive data from said body parameter measuring device and to store and/or treat said data in said calculating device.

2. A system according to claim 1, wherein the program code means cause said external calculating device to remind the user to follow a predetermined measurement schedule stored in said external calculating device by creating an audible and/or visible signal.

3. A system according to one of the claims 1 or 2, wherein the program code means cause the external calculating device to communicate with and send data to a central storage means or to a central server.

4. A system according to one of the claims 1 to 3, wherein the computer readable code is designed such as to run on a personal digital assistant.

5. A system according to one of the claims 1 to 4, wherein the body parameter measuring device is a measuring device selected from the group consisting of blood pressure monitors, body weight meters, body fat meters, cholesterol meters, glucose meters, peak flow meters and clinical thermometers.

6. A system according to one of the claims 1 to 5, wherein the program code means cause the external calculating device to display a graphic representation of the values of said body parameter.

7. A system according to one of the claims 1 to 6, wherein that body parameter measuring device has one single operation button.

8. A system according to one of the claims 1 to 7, wherein the program code means cause the external calculating device to provide an input interface for the input of patient data.

9. A system according to one of the claims 5 to 9, wherein the body parameter measuring device is a blood pressure monitor and wherein the program code means cause the external calculating device to store a plurality of subsequent pulse rate values and/or blood pressure values.

10. A system according to claim 9, wherein the program code means cause
the external calculating device to provide a user input interface for entering data relating to at least one predetermined risk factor in context with the risk of developping a disease such as a cardiovascular disease,
to store said data in a memory
to calculate a risk rate on the basis of said data and of said at least one blood pressure value or said pulse rate value
do display said risk rate as calculated.

11. A system according to claim 10, wherein said at least one risk factor is selected from the group consisting of sex and age, smoker, cholesterol level, family history and diabetes.

12. A system according to one of the claims 10 or 11, wherein the program code means cause the external calculating device to calculate a limited number of different risk rates.

13. A system according to claim 12, wherein the risk rates are NO RISK, LOW RISK, MEDIUM RISK, HIGH RISK and VERY HIGH RISK.

14. A system according to one of the claims 1 to 13, wherein the system includes the external calculating device.

15. A system according to one of the claims 1 to 14, wherein said program code allows the external calculating device to permanently store data relating to a plurality of users.

16. A system according to one of the claims 1 to 15, wherein the communication interface is an IrDA interface.

17. A blood pressure monitor, especially in a system according to one of the claims 1 to 16, with a computer program product associated therewith, wherein the computer program product has a computer usable medium having computer readable code means embodied therein, wherein after each measurement of the blood pressure the program code means cause said blood pressure monitor or an external calculating device
to store pulse rate values or blood pressure values measured in this measurement,
to form an average or averages of a plurality of pulse rate values or blood pressure values stored in said blood pressure monitor or in said external calculating device,
to compare said average or said averages with a predetermined first threshold value or with predetermined first threshold values stored in said blood pressure monitor or in said external calculating device,
to compare the values measured in this measurement with a second threshold value or with second threshold values,
to display an indication to take medicine when said average is or when said averages are above said first threshold values and/or when at least of said values is above said second threshold value.

18. A system according to one of the claims 1 to 8, wherein said body parameter measuring device is a medical thermometer and wherein said program code means cause said external calculating device to display a series of temperature values.

19. A medical thermometer, especially in a system according to claim 18,
comprising a housing and a stem projecting from said housing and a tip with a temperature sensor arranged on said stem, wherein said stem is made of a flexible material and is adapted to be wound around said housing.

20. A medical thermometer according to claim 19, wherein said medical thermometer has means for communication with an external calculating device and wherein said medical thermometer is free from a temperature display.
